SEQUENCE LISTING (6 sequences)

SEQ ID NO: 1 - DNA, 1026 bp, Bacillus licheniformis
  CDS (1)..(1026)
atgaagaaattaatcagcatcatctttatctttgtattaggggttgtcgggtcattgacagcggcggtttcggcagaagcagcttctgccttaaactcgggcaaagtaaatccgcttgccgacttcagcttaaaaggctttgccgcactaaacggcggaacaacgggcggagaaggcggtcagacggtaaccgtaacaacgggagatcagctgattgcggcattaaaaaataagaatgcaaatacgcctttaaaaatttatgtcaacggcaccattacaacatcaaatacatccgcatcaaagattgacgtcaaagacgtgtcaaacgtatcgattgtcggatcagggaccaaaggggaactcaaagggatcggcatcaaaatatggcgggccaacaacatcatcatccgcaacttgaaaattcacgaggtcgcctcaggcgataaagacgcgatcggcattgaaggcccttctaaaaacatttgggttgatcataatgagctttaccacagcctgaacgttgacaaagattactatgacggattatttgacgtcaaaagagatgcggaatatattacattctcttggaactatgtgcacgatggatggaaatcaatgctgatgggttcatcggacagcgataattacaacaggacgattacattccatcataactggtttgagaatctgaattcgcgtgtgccgtcattccgtttcggagaaggccatatttacaacaactatttcaataaaatcatcgacagcggaattaattcgaggatgggcgcgcgcatcagaattgagaacaacctctttgaaaacgccaaagatccgattgtctcttggtacagcagttcaccgggctattggcatgtatccaacaacaaatttgtaaactctaggggcagtatgccgactacctctactacaacctataatccgccatacagctactcactcgacaatgtcgacaatgtaaaatcaatcgtcaagcaaaatgccggagtcggcaaaatcaatccataa

SEQ ID NO: 2 - protein, 341 aa, Bacillus licheniformis
MKKLISIIFIFVLGVVGSLTAAVSAEAASALNSGKVNPLADFSLKGFAALNGGTTGGEGGQTVTVTTGDQLIAALKNKNANTPLKIYVNGTITTSNTSASKIDVKDVSNVSIVGSGTKGELKGIGIKIWRANNIIIRNLKIHEVASGDKDAIGIEGPSKNIWVDHNELYHSLNVDKDYYDGLFDVKRDAEYITFSWNYVHDGWKSMLMGSSDSDNYNRTITFHHNWFENLNSRVPSFRFGEGHIYNNYFNKIIDSGINSRMGARIRIENNLFENAKDPIVSWYSSSPGYWHVSNNKFVNSRGSMPTTSTTTYNPPYSYSLDNVDNVKSIVKQNAGVGKINP

SEQ ID NO: 3 - DNA, 1455 bp, Bacillus sp.
  mat_peptide (1)..(): SP722
  CDS (1)..(1455)
catcataatgggacaaatgggacgatgatgcaatactttgaatggcacttgcctaatgatgggaatcactggaatagattaagagatgatgctagtaatctaagaaatagaggtataaccgctatttggattccgcctgcctggaaagggacttcgcaaaatgatgtggggtatggagcctatgatctttatgatttaggggaatttaatcaaaaggggacggttcgtactaagtatgggacacgtagtcaattggagtctgccatccatgctttaaagaataatggcgttcaagtttatggggatgtagtgatgaaccataaaggaggagctgatgctacagaaaacgttcttgctgtcgaggtgaatccaaataaccggaatcaagaaatatctggggactacacaattgaggcttggactaagtttgattttccagggaggggtaatacatactcagactttaaatggcgttggtatcatttcgatggtgtagattgggatcaatcacgacaattccaaaatcgtatctacaaattccgaggtgatggtaaggcatgggattgggaagtagattcggaaaatggaaattatgattatttaatgtatgcagatgtagatatggatcatccggaggtagtaaatgagcttagaagatggggagaatggtatacaaatacattaaatcttgatggatttaggatcgatgcggtgaagcatattaaatatagctttacacgtgattggttgacccatgtaagaaacgcaacgggaaaagaaatgtttgctgttgctgaattttggaaaaatgatttaggtgccttggagaactatttaaataaaacaaactggaatcattctgtctttgatgtcccccttcattataatctttataacgcgtcaaatagtggaggcaactatgacatggcaaaacttcttaatggaacggttgttcaaaagcatccaatgcatgccgtaacttttgtggataatcacgattctcaacctggggaatcattagaatcatttgtacaagaatggtttaagccacttgcttatgcgcttattttaacaagagaacaaggctatccctctgtcttctatggtgactactatggaattccaacacatagtgtcccagcaatgaaagccaagattgatccaatcttagaggcgcgtcaaaattttgcatatggaacacaacatgattattttgaccatcataatataatcggatggacacgtgaaggaaataccacgcatcccaattcaggacttgcgactatcatgtcggatgggccagggggagagaaatggatgtacgtagggcaaaataaagcaggtcaagtttggcatgacataactggaaataaaccaggaacagttacgatcaatgcagatggatgggctaatttttcagtaaatggaggatctgtttccatttgggtgaaacga

SEQ ID NO: 4 - protein, 485 aa, Bacillus sp.
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 5 - DNA, 1455 bp, Bacillus sp.
  CDS (1)..(1455): AA560
  mat_peptide (1)..()
caccataatggtacgaacggcacaatgatgcagtactttgaatggtatctaccaaatgacggaaaccattggaatagattaaggtctgatgcaagtaacctaaaagataaagggatctcagcggtttggattcctcctgcatggaagggtgcctctcaaaatgatgtggggtatggtgcttatgatctgtatgatttaggagaattcaatcaaaaaggaaccattcgtacaaaatatggaacgcgcaatcagttacaagctgcagttaacgccttgaaaagtaatggaattcaagtgtatggcgatgttgtaatgaatcataaagggggagcagacgctaccgaaatggttagggcagttgaagtaaacccgaataatagaaatcaagaagtgtccggtgaatatacaattgaggcttggacaaagtttgactttccaggacgaggtaatactcattcaaacttcaaatggagatggtatcactttgatggagtagattgggatcagtcacgtaagctgaacaatcgaatttataaatttagaggtgatggaaaagggtgggattgggaagtcgatacagaaaacggtaactatgattacctaatgtatgcagatattgacatggatcacccagaggtagtgaatgagctaagaaattggggtgtttggtatacgaatacattaggccttgatggttttagaatagatgcagtaaaacatataaaatacagctttactcgtgattggattaatcatgttagaagtgcaactggcaaaaatatgtttgcggttgcggaattttggaaaaatgatttaggtgctattgaaaactatttaaacaaaacaaactggaaccattcagtctttgatgttccgctgcactataacctctataatgcttcaaaaagcggagggaattatgatatgaggcaaatatttaatggtacagtcgtgcaaagacatccaatgcatgctgttacatttgttgataatcatgattcgcaacctgaagaagctttagagtcttttgttgaagaatggttcaaaccattagcgtatgctttgacattaacacgtgaacaaggctacccttctgtattttatggagattattatggcattccaacgcatggtgtaccagcgatgaaatcgaaaattgacccgattctagaagcgcgtcaaaagtatgcatatggaagacaaaatgactacttagaccatcataatatcatcggttggacacgtgaagggaatacagcacaccccaactccggtttagctactatcatgtccgatggggcaggaggaaataagtggatgtttgttgggcgtaataaagctggtcaagtttggaccgatatcactggaaatcgtgcaggtactgttacgattaatgctgatggatggggtaatttttctgtaaatggaggatcagtttctatttgggtaaacaaa

SEQ ID NO: 6 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

(19) United States
(12) Patent Application Publication
(10) Pub. No.: US 2007/0243596 A1
(43) Pub. Date: Oct. 18, 2007

(54) Simultaneous Desizing and Scouring Process

(75) Inventors: Jiyin Liu, Raleigh, NC (US); Sonja Salmon, Raleigh, NC (US); Harm Albertus Kuildred, Beijing (CN)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 11/628,279

(22) PCT Filed: Jun. 14, 2005
(86) PCT No.: PCT/US05/20868

§ 371 Date: Nov. 30, 2006

Related U.S. Application Data
(60) Provisional application No. 60/579,714, filed on Jun. 15, 2004.

(51) Int. Cl. D06M 16/00; C12N 9/42

(52) U.S. Cl. 435/209; 435/263

(57) ABSTRACT

The present invention relates to a process for simultaneously desizing and scouring of sized fabric containing starch or starch derivatives, which process comprises treating fabric with an alkaline alpha-amylase and an alkaline scouring enzyme. The invention also relates to a composition comprising an alkaline alpha-amylase and an alkaline scouring enzyme.

### REFERENCE TO SEQUENCE LISTING

The present application contains information in the form of a sequence listing, which is appended to the application and also submitted on a data carrier accompanying this application. The content of the data carrier is fully incorporated herein by reference.

### FIELD OF THE INVENTION

The present invention relates to a process for simultaneously desizing and scouring of sized fabric. The invention also relates to a composition suitable for use in a process of the invention.

### BACKGROUND OF THE INVENTION

In the textile processing industry, alpha-amylases are traditionally used as auxiliaries in desizing processes to facilitate the removal of starch-containing size which has served as a protective coating on yarns during weaving. Complete removal of the size coating after weaving is important to ensure optimum results in the subsequent processes, in which the fabric is generally scoured, bleached, dyed and/or printed. Enzymatic starch break-down is preferred because it does not involve any harmful effect on the fibre material. In order to reduce processing cost and increase mill throughput, the desizing processing is sometimes combined with the scouring step.

WO 95/21417 suggests the use of an oxidation stabile alpha-amylase for simultaneous desizing and scouring of sized fabric.

However, it would be desirable to provide even further improved processes for simultaneous desizing and scouring.

### SUMMARY OF THE INVENTION

The present invention is directed towards providing an improved simultaneous desizing and scouring process.

In the first aspect the invention relates to a process for simultaneously desizing and scouring of sized fabric containing starch or starch derivatives, which process comprises treating the fabric with alkaline alpha-amylase and alkaline scouring enzyme.

In context of the invention the term “fabric” includes garments, fibres, yarns and other types of processed fabrics. Fabric can be constructed from fibers by weaving, knitting or non-woven operations. Weaving and knitting require yarn as the input whereas the non-woven fabric is the result of random bonding of fibers (paper can be thought of as non-woven).

Woven fabric is constructed by weaving “filling” or weft yarns between warp yarns stretched in the longitudinal direction on the loom. The warp yarns must be sized before weaving in order to lubricate and protect them from abrasion at the high speed insertion of the filling yarns during weaving. The filling yarn can be woven through the warp yarns in a “over one—under the next” fashion (plain weave) or by “over one—under two” (twill) or any other myriad of permutations. Strength, texture and pattern are related not only to the type/quality of the yarn but also the type of weave. Generally, dresses, shirts, pants, sheeting's, towels, draperies, etc. are produced from woven fabric.

Knitting is forming a fabric by joining together interlocking loops of yarn. As opposed to weaving, which is constructed from two types of yarn and has many “ends”, knitted fabric is produced from a single continuous strand of yarn. As with weaving, there are many different ways to loop yarn together and the final fabric properties are dependent both upon the yarn and the type of knit. Underwear, sweaters, socks, sport shirts, sweat shirts etc. are derived from knit fabrics.

Non-woven fabrics are sheets of fabric made by bonding and/or interlocking fibers and filaments by mechanical, thermal, chemical or solvent mediated processes. The resultant fabric can be in the form of web-like structures, laminates or films. Typical examples are disposable baby diapers, towels, wipes, surgical gowns, fibers for the “environmental friendly” fashion, filter media, bedding, roofing materials, backing for two-dimensional fabrics and many others.

According to the invention, the process may be applied to any fabric known in the art (woven, knitted, or non-woven). In particular, the process of the invention may be applied to cellulose-containing or cellulosic fabrics, such as cotton, viscose, rayon, ramie, linen, lyocell (e.g., Tencel, produced by Courtaulds Fibers), or mixtures thereof, or mixtures of any of these fibers together with synthetic fibres (e.g., polyester, polyamid, nylon) or other natural fibers, such as wool and silk., such as viscose/cotton blends, lyocell/cotton blends, viscose/wool blends, lyocell/wool blends, cotton/wool blends; flax (linen), ramie and other fabrics based on cellulose fibers, including all blends of cellulosic fibers with other fibers such as wool, polyamide, acrylic and polyester fibers, e.g., viscose/cotton/polyester blends, wool/cotton/polyester blends, flax/cotton blends etc. The process may also be used on synthetic textiles, e.g., consisting of essentially 100% polyester, polyamid, nylon, respectively. The term “wool,” means any commercially useful animal hair product, for example, wool from sheep, camel, rabbit, goat, llama, and known as merino wool, Shetland wool, cashmere wool, alpaca wool, mohair etc. and includes wool fibers and animal hair. The process of the invention can be used on wool or animal hair material in the form of top, fiber, yarn, or woven or knitted fabric.

An alkaline alpha-amylase, used in accordance with the process of the invention, may preferably be of bacterial origin, such as especially derived from a strain of Bacillus sp.

An alkaline scouring enzyme, used in accordance with the process of the invention, may be an enzyme selected from the group consisting of alkaline pectinase, cellulase, lipase, protease, or mixtures thereof.

According to the invention an enzyme is “alkaline” when the pH optimum under the conditions present during simultaneously desizing and scouring is above 7, preferably above pH 8, especially above pH 9, such as between pH 7 and 11, such as between pH 8 and 11, or between pH 9 and 11.

The term “desizing” is intended to be understood in a conventional manner, i.e., the degradation and/or removal of sizing agents from fabric, such as warp yarns in a woven fabric.

The term “scouring” is intended to be understood in a conventional manner, i.e., the removal of non-cellulosic materials such as grease, wax, protein, hemi-cellulosic material, pectin, ash, dirt and oil from fabric.

The term “simultaneously” is intended to indicate that the desizing and scouring are carried out in a single operation. This has the advantage that the washing, rinsing and other treatments normally performed between separately conducted desizing and scouring steps are no longer required. Thereby, the water and energy demand as well as the demand to different equipment to be used for each of the processes are considerably reduced. According to a preferred embodiment the process of the invention is carried out in a single bath. The scouring enzyme may be added prior to, simultaneously with or after the desizing enzyme.

The term “fabric containing starch or starch derivatives” is intended to indicate any type of fabric, in particular woven fabric prepared from a cellulose-containing material, containing starch or starch derivatives. The fabric is normally made of cotton, viscose, flax and the like. The main part of the starch or starch derivatives present on the fabric is normally size with which the yarns, normally warp yarns, have been coated prior to weaving.

Even if not specifically mentioned in connection with the process of the invention, it is to be understood that the enzymes or agent(s) is(are) used in an “effective amount”. The term “effective amount” means an amount of, e.g., alkaline alpha-amylase and alkaline scouring enzyme that is capable of providing the desired effect, i.e., desizing and scouring of the fabric, as compared to a fabric which has not been treated with said enzymes.

In a second aspect the invention relates to a composition suitable for use in a simultaneous desizing and scouring process, which composition comprises alkaline alpha-amylase and alkaline scouring enzyme.

### DETAILED DESCRIPTION OF THE INVENTION

The present invention is directed towards providing a simultaneous desizing and scouring process. According to the invention fabric may be scoured while desizing the fabric in question. The process of the invention may be carried out using traditional sizing/desizing equipment, e.g., pad systems, J-boxes, jets, jiggers, etc. No additional process equipment is needed. This is accomplished by simultaneously treating the fabric with a combination of alkaline alpha-amylase and alkaline scouring enzyme. The inventors have found that, beside the advantages obtained by carrying out desizing and scouring simultaneously (see above), other advantages are obtained as well. Examples include one or more of: reduced use of enzymes, improved desizing, improved pectin removal, improved wettability, improved whiteness, improved fabric handling, improved fabric smoothness, and reduced pilling. The results of experiments supporting the invention are shown in Table 1 after Examples 1-13.

Woven goods are the prevalent form of fabric construction. The weaving process demands a “sizing” of the warp yarn to protect it from abrasion. Starches, unmodified and modified, polyvinyl alcohol (PVA), carboxy methyl cellulose (CMC), waxes and acrylic binders, and mixtures thereof, are examples of typically used sizing agents. The sizing agent may according to the invention be a starch-based or starch derivative-based sizing agent, but may also contain one or more non-starch or starch derivative-based sizing agents. The sizing agent(s) must be removed after the weaving process as the first step in preparing the woven goods.

Further, the fabric fibers contain natural non-cellulosic impurities, which must be removed before subsequent processing steps, such as bleaching, dyeing, printing and finishing. Scouring removes much of the natural non-cellulosic impurities, including especially cuticle (mainly consisting of waxes) and primary cell wall (mainly consisting of pectin, protein and xyloglucan). A proper wax removal is necessary for obtaining a high wettability, being a measure for obtaining a good dyeing. Removal of the primary cell wall—especially the pectins—improves wax removal and ensures a more even dyeing. Further this improves the whiteness in the bleaching process. In addition, scouring can remove dirt, soils and residual manufacturing introduced materials such as spinning, coning or sizing agents.

The Process of the Invention

According to the process of the invention the sized fabric, in either rope or open width form, is brought in contact with the processing liquid (i.e., treating solution). In the case that the size (besides the starch-based or starch derivative-based sizing agent) contains PVA or CMC it is preferred to carry out the process of the invention with hot water, surfactant and mild alkali.

In accordance with the present invention desizing and scouring takes place at the same time and at conditions normally used for textile desizing.

Therefore, in the first aspect the invention relates to a process for simultaneously desizing and scouring of a sized fabric containing starch or starch derivatives, which process comprises treating the fabric with alkaline alpha-amylase and alkaline scouring enzyme.

According to the invention the sized fabrics is treated with a combination of water, alkaline alpha-amylase and alkaline scouring enzyme (as will be described in further details below), preferably in combination with one or more agents including stabilizers, surfactants, wetting agents, dispersing agent, sequestering agents and emulsifying agents, or mixtures thereof. The sized fabric is allowed to stand in the processing liquid for a “holding period” sufficiently long to accomplish the desizing and scouring. The holding period is dependent upon the type of processing regime and the temperature and can vary from 15 minutes to 2 hours, or in some cases, several days.

The processing regime can be either batch or continuous with the fabric being contacted by the liquid processing stream in open width or rope form.

Continuous operations generally use a saturator whereby an approximate equal weight of processing liquid per weight of fabric is applied to the fabric, followed by a heated dwell chamber where the chemical reaction takes place. A washing section then prepares the fabric for the next processing step. In order to ensure a high whiteness or a good wettability and resulting dyeability, the desizing and scouring enzymes and other agents must be thoroughly removed.

In one embodiment the process of the invention is a continuous process carried out at around 100° C., e.g., 90-100° C., for between 5-30 minutes at a pH in the range from 7 to 11.

Batch processes generally takes place in one processing bath, i.e., single bath, whereby the fabric is contacted with approximately 8-15 times its weight of processing liquid. After a reaction period, the processing liquid is drained, the fabric is rinsed, and the next processing step is initiated. Discontinuous PB-processes (i.e., pad-batch processes) involves a saturator whereby an approximate equal weight of processing liquid per weight of fabric is applied to the fabric, followed by a dwell period, which in the case of CPB-process (i.e., cold pad-batch process) might be one or more days. For instance, a CPB-process may be carried out at between 20-40° C. for 8-24 hours or more at a pH in the range between around 7 and 11, preferably between around 8 and 9.5. Further, a PB-process may be carried out at between 50-85° C. for 1-6 hours at a pH in the range between around 7 and 11, preferably between around 8 and 9.5.

In one embodiment the combined desizing and scouring process of the invention may be carried out using an alkaline alpha-amylase and alkaline scouring enzyme and a strong alkali, such as sodium hydroxide or related causticizing agents such as sodium carbonate, potassium hydroxide, or mixtures thereof, under conditions known in the art for desizing and scouring to be performed.

Today the recommend concentration of a commercial desizing alpha-amylase, such as AQUAZYM™ 120 L (Novozymes A/S, Denmark), lies in the range from about 180 to 240 KNU/L, which corresponding to about 180-240 KNU per kg fabric. According to the invention this concentration can be reduced.

In a preferred embodiment the alkaline alpha-amylase is present in a concentration of 0.05-150 KNU/L treating solution, preferably, 1-100 KNU/L treating solution, especially 2-20 KNU/L treating solution or 0.05-150 KNU/Kg fabric, preferably, 1-100 KNU/kg fabric, especially 2-20 KNU/kg fabric.

Further, today the recommended concentration of a commercial pectinase for scouring, such as SCOURZYME™ L (Novozymes A/S, Denmark), lies in the range of about 1500-1875 APSU/L, which corresponding to about 1500-1875 APSU per kg fabric. According to the invention this concentration can be reduced.

In a preferred embodiment the pectinase enzyme is a pectate lyase present in a concentration in the range from 1-1,500 APSU/kg fabric, preferably 10-1,200 APSU/kg fabric, especially 100-1,000 APSU/kg fabric.

Detergents

Generally an alkali stable surfactant is added to the process to enhance solubilization of hydrophobic compounds and/or prevent their redeposition back on the fabric. In the context of this invention, a detergent is synonymous with a surfactant, and it may in particular be a non-ionic surfactant, an anionic surfactant, a cationic surfactant, an ampholytic surfactant, a zwitterionic surfactant, and a semi-polar surfactant, or a mixture hereof.

The surfactant is typically present in a composition of the invention at a level from 0.1% to 60% by weight.

The surfactant is preferably formulated to be compatible with enzyme components present in the composition. In liquid or gel compositions the surfactant is most preferably formulated in such a way that it promotes, or at least does not degrade, the stability of any enzyme in these compositions.

Preferred systems to be used according to the present invention comprise as a surfactant one or more of the nonionic and/or anionic surfactants described herein.

Polyethylene, polypropylene, and polybutylene oxide condensates of alkyl phenols are suitable for use as the nonionic surfactant of the surfactant systems of the present invention, with the polyethylene oxide condensates being preferred. These compounds include the condensation products of alkyl phenols having an alkyl group containing from about 6 to about 14 carbon atoms, preferably from about 8 to about 14 carbon atoms, in either a straight chain or branched-chain configuration with the alkylene oxide. In a preferred embodiment, the ethylene oxide is present in an amount equal to from about 2 to about 25 moles, more preferably from about 3 to about 15 moles, of ethylene oxide per mole of alkyl phenol.

Commercially available nonionic surfactants of this type include Igepal™ CO-630, marketed by the GAF Corporation; and Triton™ X-45, X-114, X-100 and X-102, all marketed by the Rohm & Haas Company. These surfactants are commonly referred to as alkylphenol alkoxylates (e.g., alkyl phenol ethoxylates).

The condensation products of primary and secondary aliphatic alcohols with about 1 to about 25 moles of ethylene oxide are suitable for use as the nonionic surfactant of the nonionic surfactant system. The alkyl chain of the aliphatic alcohol can either be straight or branched, primary or secondary, and generally contains from about 8 to about 22 carbon atoms. Preferred are the condensation products of alcohols having an alkyl group containing from about 8 to about 20 carbon atoms, more preferably from about 10 to about 18 carbon atoms, with from about 2 to about 10 moles of ethylene oxide per mole of alcohol. About 2 to about 7 moles of ethylene oxide and most preferably from 2 to 5 moles of ethylene oxide per mole of alcohol are present in said condensation products. Examples of commercially available nonionic surfactants of this type include Tergitol™ 15-S-9 (The condensation product of C₁₁-C₁₅ linear alcohol with 9 moles ethylene oxide), Tergitol™ 24-L-6 NMW (the condensation product of C₁₂-C₁₄ primary alcohol with 6 moles ethylene oxide with a narrow molecular weight distribution), both marketed by Union Carbide Corporation; Neodol™ 45-9 (the condensation product of C₁₄-C₁₅ linear alcohol with 9 moles of ethylene oxide), Neodol™ 23-3 (the condensation product of C₁₂-C₁₃ linear alcohol with 3.0 moles of ethylene oxide), Neodol™ 45-7 (the condensation product of C₁₄-C₁₅ linear alcohol with 7 moles of ethylene oxide), Neodol™ 45-5 (the condensation product of C₁₄-C₁₅ linear alcohol with 5 moles of ethylene oxide) marketed by Shell Chemical Company, Kyro™ EOB (the condensation product of C₁₃-C₁₅ alcohol with 9 moles ethylene oxide), marketed by The Procter & Gamble Company, and Genapol LA 050 (the condensation product of C₁₂-C₁₄ alcohol with 5 moles of ethylene oxide) marketed by Hoechst. Preferred range of HLB in these products is from 8-11 and most preferred from 8-10.

Also useful as the nonionic surfactant of the surfactant system are alkylpolysaccharides disclosed in U.S. Pat. No. 4,565,647, having a hydrophobic group containing from about 6 to about 30 carbon atoms, preferably from about 10 to about 16 carbon atoms and a polysaccharide, e.g., a polyglycoside, hydrophilic group containing from about 1.3 to about 10, preferably from about 1.3 to about 3, most preferably from about 1.3 to about 2.7 saccharide units. Any reducing saccharide containing 5 or 6 carbon atoms can be used, e.g., glucose, galactose and galactosyl moieties can be substituted for the glucosyl moieties (optionally the hydrophobic group is attached at the 2-, 3-, 4-, etc. positions thus giving a glucose or galactose as opposed to a glucoside or galactoside). The intersaccharide bonds can be, e.g., between the one position of the additional saccharide units and the 2-, 3-, 4-, and/or 6-positions on the preceding saccharide units.

The preferred alkylpolyglycosides have the formula
R²O(CₙH₂ₙO)ₜ(glycosyl)ₓ
wherein R² is selected from the group consisting of alkyl, alkylphenyl, hydroxyalkyl, hydroxyalkylphenyl, and mixtures thereof in which the alkyl groups contain from about 10 to about 18, preferably from about 12 to about 14, carbon atoms; n is 2 or 3, preferably 2; t is from 0 to about 10, preferably 0; and x is from about 1.3 to about 10, preferably from about 1.3 to about 3, most preferably from about 1.3 to about 2.7. The glycosyl is preferably derived from glucose. To prepare these compounds, the alcohol or alkylpolyethoxy alcohol is formed first and then reacted with glucose, or a source of glucose, to form the glucoside (attachment at the 1-position). The additional glycosyl units can then be attached between their 1-position and the preceding glycosyl units 2-, 3-, 4-, and/or 6-position, preferably predominantly the 2-position.

The condensation products of ethylene oxide with a hydrophobic base formed by the condensation of propylene oxide with propylene glycol are also suitable for use as the additional nonionic surfactant system. The hydrophobic portion of these compounds will preferably have a molecular weight from about 1500 to about 1800 and will exhibit water insolubility. The addition of polyoxyethylene moieties to this hydrophobic portion tends to increase the water solubility of the molecule as a whole, and the liquid character of the product is retained up to the point where the polyoxyethylene content is about 50% of the total weight of the condensation product, which corresponds to condensation with up to about 40 moles of ethylene oxide. Examples of compounds of this type include certain of the commercially available Pluronic™ surfactants, marketed by BASF.

Also suitable for use as the nonionic surfactant of the nonionic surfactant system are the condensation products of ethylene oxide with the product resulting from the reaction of propylene oxide and ethylenediamine. The hydrophobic moiety of these products consists of the reaction product of ethylenediamine and excess propylene oxide, and generally has a molecular weight of from about 2,500 to about 3,000. This hydrophobic moiety is condensed with ethylene oxide to the extent that the condensation product contains from about 40% to about 80% by weight of polyoxyethylene and has a molecular weight of from about 5,000 to about 11,000. Examples of this type of nonionic surfactant include certain of the commercially available Tetronic™ compounds, marketed by BASF.

Preferred for use as the nonionic surfactant of the surfactant system are polyethylene oxide condensates of alkyl phenols, condensation products of primary and secondary aliphatic alcohols with from about 1 to about 25 moles of ethyleneoxide, alkylpolysaccharides, and mixtures hereof. Most preferred are C₈-C₁₄ alkyl phenol ethoxylates having from 3 to 15 ethoxy groups and C₈-C₁₈ alcohol ethoxylates (preferably C₁₀ avg.) having from 2 to 10 ethoxy groups, and mixtures thereof.

Highly preferred nonionic surfactants are polyhydroxy fatty acid amide surfactants of the formula 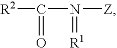
wherein R¹ is H, or R¹ is C₁₋₄ hydrocarbyl, 2-hydroxyethyl, 2-hydroxypropyl or a mixture thereof, R² is C₅₋₃₁ hydrocarbyl, and Z is a polyhydroxyhydrocarbyl having a linear hydrocarbyl chain with at least 3 hydroxyls directly connected to the chain, or an alkoxylated derivative thereof. Preferably, R¹ is methyl, R² is straight C₁₁₋₁₅ alkyl or C₁₆₋₁₈ alkyl or alkenyl chain such as coconut alkyl or mixtures thereof, and Z is derived from a reducing sugar such as glucose, fructose, maltose or lactose, in a reductive amination reaction.

Highly preferred anionic surfactants include alkyl alkoxylated sulfate surfactants. Examples hereof are water soluble salts or acids of the formula RO(A)ₘSO₃M wherein R is an unsubstituted C₁₀-C-₂₄ alkyl or hydroxyalkyl group having a C₁₀-C₂₄ alkyl component, preferably a C₁₂-C₂₀ alkyl or hydroxyalkyl, more preferably C₁₂-C₁₈ alkyl or hydroxyalkyl, A is an ethoxy or propoxy unit, m is greater than zero, typically between about 0.5 and about 6, more preferably between about 0.5 and about 3, and M is H or a cation which can be, for example, a metal cation (e.g., sodium, potassium, lithium, calcium, magnesium, etc.), ammonium or substituted-ammonium cation. Alkyl ethoxylated sulfates as well as alkyl propoxylated sulfates are contemplated herein. Specific examples of substituted ammonium cations include methyl-, dimethyl, trimethyl-ammonium cations and quaternary ammonium cations such as tetramethyl-ammonium and dimethyl piperdinium cations and those derived from alkylamines such as ethylamine, diethylamine, triethylamine, mixtures thereof, and the like. Exemplary surfactants are C₁₂-C₁₈ alkyl polyethoxylate (1.0) sulfate (C₁₂-C₁₈E(1.0)M), C₁₂-C₁₈ alkyl polyethoxylate (2.25) sulfate (C₁₂-C₁₈(2.25)M, and C₁₂-C₁₈ alkyl polyethoxylate (3.0) sulfate (C₁₂-C₁₈E(3.0)M), and C₁₂-C₁₈ alkyl polyethoxylate (4.0) sulfate (C₁₂-C₁₈E(4.0)M), wherein M is conveniently selected from sodium and potassium.

Suitable anionic surfactants to be used are alkyl ester sulfonate surfactants including linear esters of C₈-C₂₀ carboxylic acids (i.e., fatty acids) which are sulfonated with gaseous SO₃ according to “The Journal of the American Oil Chemists Society”, 52 (1975), pp. 323-329. Suitable starting materials would include natural fatty substances as derived from tallow, palm oil, etc.

The preferred alkyl ester sulfonate surfactant comprises alkyl ester sulfonate surfactants of the structural formula: 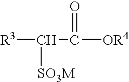
wherein R³ is a C₈-C₂₀ hydrocarbyl, preferably an alkyl, or combination thereof, R⁴ is a C₁-C₆ hydrocarbyl, preferably an alkyl, or combination thereof, and M is a cation which forms a water soluble salt with the alkyl ester sulfonate. Suitable salt-forming cations include metals such as sodium, potassium, and lithium, and substituted or unsubstituted ammonium cations, such as monoethanolamine, diethonolamine, and triethanolamine. Preferably, R³ is C₁₀-C₁₆ alkyl, and R⁴ is methyl, ethyl or isopropyl. Especially preferred are the methyl ester sulfonates wherein R₃ is C₁₀-C₁₆ alkyl.

Other suitable anionic surfactants include the alkyl sulfate surfactants which are water soluble salts or acids of the formula ROSO₃M wherein R preferably is a C₁₀-C₂₄ hydrocarbyl, preferably an alkyl or hydroxyalkyl having a C₁₀-C₂₀ alkyl component, more preferably a C₁₂-C₁₈ alkyl or hydroxyalkyl, and M is H or a cation, e.g., an alkali metal cation (e.g., sodium, potassium, lithium), or ammonium or substituted ammonium (e.g., methyl-, dimethyl-, and trimethyl ammonium cations and quaternary ammonium cations such as tetramethyl-ammonium and dimethyl piperdinium cations and quaternary ammonium cations derived from alkylamines such as ethylamine, diethylamine, triethylamine, and mixtures thereof, and the like). Typically, alkyl chains of C₁₂-C₁₆ are preferred for lower wash temperatures (e.g. below about 50° C.) and C₁₆-C₁₈ alkyl chains are preferred for higher wash temperatures (e.g. above about 50° C.).

Other anionic surfactants useful for detersive purposes include salts (including, for example, sodium, potassium, ammonium, and substituted ammonium salts such as mono- di- and triethanolamine salts) of soap, C₈-C₂₂ primary or secondary alkanesulfonates, C₈-C₂₄ olefinsulfonates, sulfonated polycarboxylic acids prepared by sulfonation of the pyrolyzed product of alkaline earth metal citrates, e.g., as described in British patent specification No. 1,082,179, C₈-C₂₄ alkylpolyglycolethersulfates (containing up to 10 moles of ethylene oxide); alkyl glycerol sulfonates, fatty acyl glycerol sulfonates, fatty oleyl glycerol sulfates, alkyl phenol ethylene oxide ether sulfates, paraffin sulfonates, alkyl phosphates, isethionates such as the acyl isethionates, N-acyl taurates, alkyl succinamates and sulfosuccinates, monoesters of sulfosuccinates (especially saturated and unsaturated C₁₂-C₁₈ monoesters) and diesters of sulfosuccinates (especially saturated and unsaturated C₆-C₁₂ diesters), acyl sarcosinates, sulfates of alkylpolysaccharides such as the sulfates of alkylpolyglucoside (the nonionic nonsulfated compounds being described below), branched primary alkyl sulfates, and alkyl polyethoxy carboxylates such as those of the formula RO(CH₂CH₂O)ₖ—CH₂COO-M+ wherein R is a C₈-C₂₂ alkyl, k is an integer from 1 to 10, and M is a soluble salt forming cation. Resin acids and hydrogenated resin acids are also suitable, such as rosin, hydrogenated rosin, and resin acids and hydrogenated resin acids present in or derived from tall oil.

Alkylbenzene sulfonates are highly preferred. Especially preferred are linear (straight-chain) alkyl benzene sulfonates (LAS) wherein the alkyl group preferably contains from 10 to 18 carbon atoms.

Further examples are described in “Surface Active Agents and Detergents” (Vol. I and II by Schwartz, Perrry and Berch). A variety of such surfactants are also generally disclosed in U.S. Pat. No. 3,929,678 (Column 23, line 58 through Column 29, line 23, herein incorporated by reference).

When included therein the compositions of the present invention typically comprise from about 1% to about 40%, preferably from about 3% to about 20% by weight of such anionic surfactants.

The compositions of the present invention may also contain cationic, ampholytic, zwitterionic, and semi-polar surfactants, as well as the nonionic and/or anionic surfactants other than those already described herein.

Cationic detersive surfactants suitable for use in the compositions of the present invention are those having one long-chain hydrocarbyl group. Examples of such cationic surfactants include the ammonium surfactants such as alkyltrimethylammonium halogenides, and those surfactants having the formula:
[R²(OR³)_{y}][R⁴(OR³)_{y}]₂R⁵N+X−
wherein R² is an alkyl or alkyl benzyl group having from about 8 to about 18 carbon atoms in the alkyl chain, each R³ is selected form the group consisting of —CH₂CH₂—, —CH₂CH(CH₃)—, —CH₂CH(CH₂OH)—, —CH₂CH₂CH₂—, and mixtures thereof; each R⁴ is selected from the group consisting of C₁-C₄ alkyl, C₁-C₄ hydroxyalkyl, benzyl ring structures formed by joining the two R⁴ groups, —CH₂CHOHCHOHCOR⁶CHOHCH₂OH, wherein R⁶ is any hexose or hexose polymer having a molecular weight less than about 1000, and hydrogen when y is not 0; R⁵ is the same as R⁴ or is an alkyl chain, wherein the total number of carbon atoms or R² plus R⁵ is not more than about 18; each y is from 0 to about 10, and the sum of the y values is from 0 to about 15; and X is any compatible anion.

Highly preferred cationic surfactants are the water soluble quaternary ammonium compounds useful in the present composition having the formula:
R₁R₂R₃R₄N⁺X⁻ (i)
wherein R₁ is C₈-C₁₆ alkyl, each of R₂, R₃ and R₄ is independently C₁-C₄ alkyl, C₁-C₄ hydroxy alkyl, benzyl, and —(C₂H₄₀)ₓH where x has a value from 2 to 5, and X is an anion. Not more than one of R₂, R₃ or R₄ should be benzyl.

The preferred alkyl chain length for R₁ is C₁₂-C₁₅, particularly where the alkyl group is a mixture of chain lengths derived from coconut or palm kernel fat or is derived synthetically by olefin build up or OXO alcohols synthesis.

Preferred groups for R₂R₃ and R₄ are methyl and hydroxyethyl groups and the anion X may be selected from halide, methosulphate, acetate and phosphate ions.

Examples of suitable quaternary ammonium compounds of formulae (i) for use herein are:

coconut trimethyl ammonium chloride or bromide;

coconut methyl dihydroxyethyl ammonium chloride or bromide;

decyl triethyl ammonium chloride;

decyl dimethyl hydroxyethyl ammonium chloride or bromide;

C₁₂₋₁₅ dimethyl hydroxyethyl ammonium chloride or bromide;

coconut dimethyl hydroxyethyl ammonium chloride or bromide;

myristyl trimethyl ammonium methyl sulphate;

lauryl dimethyl benzyl ammonium chloride or bromide;

lauryl dimethyl(ethenoxy)₄ ammonium chloride or bromide;

choline esters (compounds of formula (i) wherein R₁ is 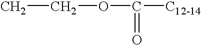
alkyl and R₂R₃R₄ are methyl).

di-alkyl imidazolines [compounds of formula (i)].

Other cationic surfactants useful herein are also described in U.S. Pat. No. 4,228,044 and in EP 000 224.

When included therein, the compositions of the present invention typically comprise 20 from 0.2% to about 25%, preferably from about 1% to about 8% by weight of such cationic surfactants.

Ampholytic surfactants are also suitable for use in the compositions of the present invention. These surfactants can be broadly described as aliphatic derivatives of secondary or tertiary amines, or aliphatic derivatives of heterocyclic secondary and tertiary amines in which the aliphatic radical can be straight or branched-chain. One of the aliphatic substituents contains at least about 8 carbon atoms, typically from about 8 to about 18 carbon atoms, and at least one contains an anionic water-solubilizing group, e.g., carboxy, sulfonate, sulfate. See U.S. Pat. No. 3,929,678 (column 19, lines 18-35) for examples of ampholytic surfactants.

When included therein, the compositions of the present invention typically comprise from 0.2% to about 15%, preferably from about 1% to about 10% by weight of such ampholytic surfactants.

Zwitterionic surfactants are also suitable for use in the composition of the invention. These surfactants can be broadly described as derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 (column 19, line 38 through column 22, line 48) for examples of zwitterionic surfactants.

When included therein, the compositions of the present invention typically comprise from 0.2% to about 15%, preferably from about 1% to about 10% by weight of such zwitterionic surfactants.

Semi-polar nonionic surfactants are a special category of nonionic surfactants which include water-soluble amine oxides containing one alkyl moiety of from about 10 to about 18 carbon atoms and 2 moieties selected from the group consisting of alkyl groups and hydroxyalkyl groups containing from about 1 to about 3 carbon atoms; watersoluble phosphine oxides containing one alkyl moiety of from about 10 to about 18 carbon atoms and 2 moieties selected from the group consisting of alkyl groups and hydroxyalkyl groups containing from about 1 to about 3 carbon atoms; and water-soluble sulfoxides containing one alkyl moiety from about 10 to about 18 carbon atoms and a moiety selected from the group consisting of alkyl and hydroxyalkyl moieties of from about 1 to about 3 carbon atoms.

Semi-polar nonionic detergent surfactants include the amine oxide surfactants having the formula: 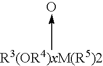
wherein R³ is an alkyl, hydroxyalkyl, or alkyl phenyl group or mixtures thereof containing from about 8 to about 22 carbon atoms; R⁴ is an alkylene or hydroxyalkylene group containing from about 2 to about 3 carbon atoms or mixtures thereof; x is from 0 to about 3: and each R⁵ is an alkyl or hydroxyalkyl group containing from about 1 to about 3 carbon atoms or a polyethylene oxide group containing from about 1 to about 3 ethylene oxide groups. The R⁵ groups can be attached to each other, e.g., through an oxygen or nitrogen atom, to form a ring structure.

These amine oxide surfactants in particular include C₁₀-C₁₈ alkyl dimethyl amine oxides and C₈-C₁₂ alkoxy ethyl dihydroxy ethyl amine oxides.

When included therein, the composition of the present invention typically comprises from 0.2% to about 15%, preferably from about 1% to about 10% by weight of such semi-polar nonionic surfactants.

Enzymes

Amylases

Any alkaline alpha-amylase may be used according to the invention. An amylase is “alkaline” in context of the present invention when the pH optimum under the conditions present during simultaneously desizing and scouring is above 7, preferably above 8, especially above 9.

Suitable alpha-amylases include those of bacterial or fungal origin. Chemically or genetically modified mutants (variants) are included. A preferred alkaline alpha-amylase is derived from a strain of Bacillus, such as Bacillus licheniformis, Bacillus amyloliquefaciens, Bacillus stearothermophilus, Bacillus subtilis, or other Bacillus sp., such as Bacillus sp. NCIB 12289, NCIB 12512, NCIB 12513, DSM 9375, DSMZ no. 12649, KSM AP1378 (WO 97/00324), KSM K36 or KSM K38 (EP 1,022,334). Preferred are the Bacillus sp. alpha-amylases disclosed in WO 95/26397 as SEQ ID NOS. 1 and 2 (i.e., SEQ ID NO: 4 herein), respectively, the alpha-amylase disclosed as SEQ ID NO: 2 in WO 00/60060 (i.e., SEQ ID NO: 6 herein), and the #707 alpha-amylase disclosed by Tsukamoto et al., Biochemical and Biophysical Research Communications, Vol. 151, pp. 25-31 (1988).

Commercially available alkaline alpha-amylase products or products comprising alpha-amylases include product sold under the following tradenames: NATALASE™, STAINZYME™ (Novozymes A/S), BIOAMYLASE—D(G), BIOAMYLASE™ L (Biocon India Ltd.), KEMZYM™ AT 9000 (Biozym Ges. m.b.H, Austria), PURASTAR™ ST, PURASTAR™ HPAmL, PURAFECT™ OxAm, RAPIDASE™ TEX (Genencor Int. Inc, USA), KAM (KAO, Japan)

In a specific embodiment of the invention the alkaline alpha-amylase is the alpha-amylase having an amino acid sequence of SEQ ID NO: 4 or the alpha-amylase having an amino acid sequence of SEQ ID NO: 6, or an alpha-amylase having a degree of identity of at least 60%, preferably at least 70%, more preferred at least 80%, even more preferred at least 90%, such as at least 95%, at least 96%, at least 97%, at least 98% or at least 99% to any of the sequences of SEQ ID NO: 4 or 6.

For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10, and gap length penalty of 10. Pairwise alignment parameters were Ktuple=1, gap penalty=3, windows=5, and diago-nals=5].

In a preferred embodiment the parent alpha-amylase has one or more deletions in positions D183 and G184, preferably wherein said alpha-amylase variant further has a substitution in position N195F (using SEQ ID NO: 4 numbering).

In another preferred embodiment the parent alpha-amylase has one or more of the following deletions/substitutions: Delta (R81−G182); Delta (D183−G184); Delta (D183−G184)+N195F; R181Q+N445Q+K446N; Delta (D183−G184)+R181Q, Delta (D183−G184) and one or more of the following substitutions: R118K, N195F, R320K, R458K, especially wherein the variant has the following mutations: Delta(D183+G184)+R118K+N195F+R320K+R458K (using SEQ ID NO: 6 numbering).

In another preferred embodiment the alkaline alpha-amylase is the alpha-amylase shown in SEQ ID NO: 6 further comprising one or more of the following substitutions M9L, M202L, V214T, M323T, M382Y, E345R or the A560 alpha-amylase with all of the following substitutions: M9L, M202L, V214T, M323T, M382Y or M9L, M202L, V214T, M323T and E345R.

In an embodiment of the process of the invention the alkaline alpha-amylase may preferably be present in a concentration of 0.05-150 KNU/L treating solution, preferably, 1-100 KNU/L treating solution, especially 2-20 KNU/L treating solution or 0.05-150 KNU/Kg fabric, preferably, 1-100 KNU/kg fabric, especially 2-20 KNU/kg fabric

Alkaline Scouring Enzymes

Any alkaline scouring enzyme may be used according to the invention. The alkaline scouring enzyme may be an alkaline enzyme selected from the group consisting of pectinase, cellulase, lipase, protease, xyloglucanase, cutinase and a mixture thereof. A scouring enzyme is “alkaline” in context of the present invention when the pH optimum under the conditions present during simultaneously desizing and scouring is above 7, preferably above 8, especially above 9.

In a preferred embodiment the alkaline pectinase is a pectate lyase, a pectine lyase, a polygalacturonase, or a polygalacturonate lyase.

Pectinase

The term “pectinase” is intended to include any alkaline pectinase enzyme. Pectinases are a group of enzymes that hydrolyse glycosidic linkages of pectic substances mainly poly-1,4-alpha-D-galacturonide and its derivatives (see reference Sakai et al., Pectin, pectinase and propectinase: production, properties and applications, in: Advances in Applied Microbiology, Vol. 39, pp. 213-294 (1993)) which enzyme is understood to include a mature protein or a precursor form thereof, or a functional fragment thereof, which essentially has the activity of the full-length enzyme. Furthermore, the term pectinase enzyme is intended to include homologues or analogues of such enzymes.

Preferably the alkaline pectinase is an enzyme which catalyzes the random cleavage of alpha-1,4-glycosidic linkages in pectic acid also called polygalacturonic acid by transelimination such as the enzyme class polygalacturonate lyase (EC 4.2.2.2) (PGL) also known as poly(1,4-alpha-D-galacturonide) lyase also known as pectate lyase. Also preferred is a pectinase enzyme which catalyzes the random hydrolysis of alpha-1,4-glycosidic linkages in pectic acid such as the enzyme class polygalacturonase (EC 3.2.1.15) (PG) also known as endo-PG. Also preferred is a pectinase enzyme such as polymethylgaicturonate lyase (EC 4.2.2.10) (PMGL), also known as Endo-PMGL, also known as poly(methyoxygalacturonide)lyase also known as pectin lyase which catalyzes the random cleavage of alpha-1,4-glycosidic linkages of pectin. Other preferred pectinases are galactanases (EC 3.2.1.89), arabinanases (EC 3.2.1.99), pectin esterases (EC 3.1.1.11), and mannanases (EC 3.2.1.78).

The enzyme is preferably derived from a micro-organism, preferably from a bacterium, an archea or a fungus, especially from a bacterium such as a bacterium belonging to the genus Bacillus, preferably to an alkalophilic Bacillus strain which may be selected from the group consisting of the species Bacillus licheniformis and highly related Bacillus species in which all species are at least 90% homologous (identical) to Bacillus licheniformis based on aligned 16S rDNA sequences. Specific examples of such species are the species Bacillus licheniformis, Bacillus alcalophilus, Bacillus pseudoalcalophilus, and Bacillus clarkii. A specific and highly preferred example is the strain Bacillus licheniformis, ATCC 14580 (U.S. Pat. No. 6,284,524). Other useful pectate lyases are derivable from the species Bacillus agaradhaerens, especially from the strain deposited as NCIMB 40482; and from the species Bacillus subtilis, Bacillus stearothermophilus, Bacillus pumilus, Bacillus cohnii, Bacillus pseudoalcalophilus, Erwinia sp. 9482, especially the strain FERM BP-5994, and Paenibacillus polymyxa.

The pectinase may be a component occurring in an enzyme system produced by a given micro-organism, such an enzyme system mostly comprising several different pectinase components including those identified above.

Alternatively, the pectinase may be a single component, i.e., a component essentially free of other pectinase enzymes which may occur in an enzyme system produced by a given micro-organism, the single component typically being a recombinant component, i.e., produced by cloning of a DNA sequence encoding the single component and subsequent cell transformed with the DNA sequence and expressed in a host. Such useful recombinant enzymes, especially pectate lyases, pectin lyases and polygalacturonases are described in detail in, e.g., WO 99/27083 and WO 99/27084 (from Novozymes A/S) which are hereby incorporated by reference in their entirety including the sequence listings. The host is preferably a heterologous host, but the host may under certain conditions also be the homologous host.

In a preferred embodiment the pectate lyase used according to the invention is derived from the genus Bacillus, preferably the species Bacillus licheniformis Bacillus alcalophilus, Bacillus pseudoalcalophilus, and Bacillus clarkia, especially the species Bacillus licheniformis, ATCC 14580.

In an even more preferred embodiment the pectate lyase is the mature pectase lyase in SEQ ID NO: 2 herein derived from a strain of Bacillus licheniformis. The pectate lyase is also disclosed in U.S. Pat. No. 6,284,524, which is hereby incorporated by reference.

The pectinase, such as especially pectate lyase, may preferably be present in a concentration in the range from 1-1,500 APSU/kg fabric, preferably 10-1,200 APSU/kg fabric, especially 100-1,000 APSU/kg fabric

Commercially available alkaline pectate lyases include BIOPREP™ and SCOURZYME™ L from Novozymes A/S, Denmark.

Proteases

Any protease suitable for use in alkaline solutions can be used. Suitable proteases include those of animal, vegetable or microbial origin. Microbial origin is preferred. Chemically or genetically modified mutants are included. The protease may be a serine protease, preferably an alkaline microbial protease or a trypsin-like protease. Examples of alkaline proteases are subtilisins, especially those derived from Bacillus, preferably Bacillus lentus or Bacillus clausli, e.g., subtilisin Novo, subtilisin Carlsberg, subtilisin 309, subtilisin 147 and subtilisin 168 (described in WO 89/06279).

Preferred commercially available protease enzymes include those sold under the trade names ALCALASE™, SAVINASE™ 16 L Type Ex, PRIMASE™, DURAZYM™, and ESPERASE™ (Novozymes A/S, Denmark), those sold under the tradename OPTICLEAN™, OPTIMASE™, PROPARASE™, PURAFECT™, PURAPECT™ MA and PURAPECT™ OX, PURAFECT™ OX-1 and PURAFECT™ OX-2 by Genencor International Inc., (USA).

In an embodiment of the process of the invention a In an embodiment of the process of the invention protease may be present in a concentration from 0.001-10 KNPU/L, preferably 0.1-1 KNPU/L, especially around 0.3 KNPU/L or 0.001-10 KNPU/kg fabric, preferably 0.1-1 KNPU/kg fabric, especially around 0.3 KNPU/kg fabric.

Lipases

Any lipase suitable for use in alkaline solutions can be used. Suitable lipases include those of bacterial or fungal origin. Chemically or genetically modified mutants are included. Examples of useful lipases include a Humicola lanuginosa lipase, e.g., as described in EP 258 068 and EP 305 216, a Rhizomucor miehei lipase, e.g., as described in EP 238 023, a Candida lipase, such as a C. antarctica lipase, e.g., the C. antarctica lipase A or B described in EP 214 761, a Pseudomonas lipase such as a P. alcaligenes and P. pseudoalcaligenes lipase, e.g., as described in EP 218 272, a P. cepacia lipase, e.g., as described in EP 331 376, a P. stutzeri lipase, e.g., as disclosed in GB 1,372,034, a P. fluorescens lipase, a Bacillus lipase, e.g., a B. subtilis lipase (Dartois et al., Biochemica et Biophysica Acta 1131, 253-260 (!993)), a B. stearothermophilus lipase (JP 64/744992) and a B. pumilus lipase (WO 91/16422).

Furthermore, a number of cloned lipases may be useful, including the Penicillium camembedtii lipase described by Yamaguchi et al., Gene 103, 61-67 (1991)), the Geotricum candidum lipase (Schimada, Y. et al., J. Biochem., Vol. 106, pp. 383-388 (1989)), and various Rhizopus lipases such as a R. delemar _lipase (Hass, M. J et al., Gene, Vol. 109, pp. 117-113 (1991)), a R. niveus lipase (Kugimiya et al., Biosci. Biotech. Biochem., Vol. 56, pp. 716-719 (1992)) and a R. oryzae lipase.

Especially suitable are lipases such as M1 LIPASE™, LUMA FAST™ and LIPOMAX™ (Genencor International Inc, USA), LIPOLASE™ and LIPOLASE ULTRA™, SP735 (Novozymes A/S, Denmark), and LIPASE P “Amano” (Amano Pharmaceutical Co. Ltd.).

In an embodiment of the process of the invention a lipase enzyme may be present in a concentration from 0.01-100 LU/L treating solution, preferably 1-10 LU/L treating solution, especially around 1 LU/L treating solution or from 0.01-100 LU/kg fabric, preferably 1-10 LU/kg fabric, especially around 1 LU/kg fabric.

Cellulases

In the present context, the term “cellulase or “cellulolytic enzyme” refers to an enzyme, which catalyzes the degradation of cellulose to glucose, cellobiose, triose and other cellooligosaccharides. Cellulose is a polymer of glucose linked by beta-1,4-glucosidic bonds. Cellulose chains form numerous intra- and intermolecular hydrogen bonds, which result in the formation of insoluble cellulose microfibrils. Microbial hydrolysis of cellulose to glucose involves the following three major classes of cellulases: endo-1,4-beta-glucanases (EC 3.2.1.4), which cleave beta-1,4-glucosidic links randomly throughout cellulose molecules; cellobiohydrolases (EC 3.2.1.91) (exoglucanases), which digest cellulose from the nonreducing end; and beta-glucosidases (EC 3.2.1.21), which hydrolyse cellobiose and low-molecular-mass cellodextrins to release glucose. Most cellulases consist of a cellulose-binding domain (CBD) and a catalytic domain (CAD) separated by a linker rich in proline and hydroxy amino acid residues. In the specification and claims, the term “endoglucanase” is intended to denote enzymes with cellulolytic activity, especially endo-1,4-beta-glucanase activity, which are classified in EC 3.2.1.4 according to the Enzyme Nomenclature (1992) and are capable of catalyzing (endo)hydrolysis of 1,4-beta-D-glucosidic linkages in cellulose, lichenin and cereal beta-D-glucans including 1,4-linkages in beta-D-glucans also containing 1,3-linkages. Any cellulase suitable for use in alkaline solutions can be used. Suitable cellulases include those of bacterial or fungal origin. Chemically or genetically modified mutants are included. Suitable cellulases are disclosed in U.S. Pat. No. 4,435,307, which discloses fungal cellulases produced from Humicola insolens. Especially suitable cellulases are the cellulases having colour care benefits. Examples of such cellulases are cellulases described in European patent application No. 0 495 257, WO 91/17243 and WO 96/29397.

In a preferred embodiment alkaline cellulase is an alkaline endoglucanase, preferably a Humicola endoglucanase, especially a Humicola insolens endoglucanase, even more preferred an EG I or EG V endoglucanase from Humicolas insolens DSM 1800, or a variant thereof, or Thielavia endoglucanase, preferably a Thielavia terrestris endoglucanase, or a variant thereof.

Commercially available cellulases include CELLUZYME™ and DENIMAX™ 399S produced by a strain of Humicola insolens (Novozymes A/S), and KAC-500(B)™ (Kao Corporation).

In an embodiment of the process of the invention the cellulase may be used in a concentration in the range from 0.001-10 g enzyme protein/L treating solution, preferably 0.005-5 g enzyme protein/L treating solution, especially 0.01-3 g enzyme protein/L solution or from 0.001-10 g enzyme protein/kg fabric, preferably 0.005-5 g enzyme protein/kg fabric, especially 0.01-3 g enzyme protein/kg fabric. In an embodiment the cellulose is used in a concentration of from 0.1-1,000 ECU/g fabric, preferably 0.5-200 ECU/g fabric, especially 1-500 ECU/g fabric.

Cutinase

A cutinase is an enzyme capable of degrading cutin, cf. e.g. Lin T S & Kolattukudy P E, J. Bacteriol. 1978 133 (2) 942-951, Cutinases, for instance, differs from classical lipases in that no measurable activation around the critical micelle concentration (CMC) of the tributyrine substrate is observed. Also, cutinases are considered belonging to a class of serine esterases. The cutinase may also be a cutinase derived from Humicola insolens disclosed in WO 96/13580. The cutinase may be a variant such as one or the variants disclosed in WO 00/34450 and WO 01/92502 which is hereby incorporated by reference.

Examples of cutinases are those derived from Humicola insolens (U.S. Pat. No. 5,827,719); from a strain of Fusarium, e.g. F. roseum culmorum, or particularly F. solani pisi (WO 90/09446; WO 94/14964, WO 94/03578). The cutinase may also be derived from a strain of Rhizoctonia, e.g. R. solani, or a strain of Alternaria, e.g. A. brassicicola (WO 94/03578), or variants thereof such as those described in WO 00/34450, or WO 01/92502. The cutinase may also be of bacterial origin, such as a strain of Pseudomonas, preferably Pseudomonas mendocina disclosed in WO 01/34899.

The cutinase may be added in a concentration of 0.001-25,000 micrograms enzyme protein/gram fabric, preferably 0.01-10,000 micrograms enzyme protein/g fabric, especially 0.05-1,000 micrograms enzyme protein/g fabric.

Xyloglucanase

A xyloglucanase is a xyloglucan specific enzyme capable of catalyzing the solubilization of xyloglucan to xyloglucan oligosaccharides. According to IUBMB Enzyme Nomenclature (2003) a xyloglucanase is classified as EC 3.2.1.151. Pauly et al. Glycobiology 9 (1999) p. 93-100, discloses a xyloglucan specific endo-beta-1,4-glucanase from Aspergillus aculeatus. A xyloglucanase used according to the invention may be derived from micro-organisms such as fungi or bacteria. Examples of useful xyloglucanases are family 12 xyloglucan hydrolyzing endoglucanases, in particular family 12 xyloglucan hydrolyzing endoglucanases, obtained from, e.g., Aspergillus aculeatus as described in WO 94/14953. Another useful example is a xyloglucanase produced by Trichoderma, especially EGIII. The xyloglucanase may also be derived from a bacterium from the genus Bacillus, including Bacillus licheniformis, Bacillus agaradharens or Bacillus firmus. The xyloglucanase may also be an endoglucanase with xyloglucanase activity and low activity towards insoluble cellulose and high activity towards soluble cellulose, e.g., family 7 endoglucanases obtained from, e.g., Humicola insolens.

The xyloglucanase may be added in a concentration of 0.001-25,000 micrograms enzyme protein/gram fabric, preferably 0.01-10,000 micrograms enzyme protein/g fabric, more preferably 0.05-1,000 micrograms enzyme protein/g fabric, in particular 0.5-500 micrograms enzyme protein/gram fabric.

Composition of the Invention

In the second aspect the invention relates to a composition suitable for use in the process of the invention. The composition may be a solid or liquid (aqueous) composition and may be a concentrated composition or a ready-to-use composition.

Thus, in this aspect the invention relates to a composition comprising an alkaline alpha-amylase and an alkaline scouring enzyme.

The enzymes comprised may preferably be the ones mentioned in the “Enzymes” section above.

In a preferred embodiment the alkaline alpha-amylase derived from a strain of Bacillus sp., preferably from a strain of B. Icheniformis, B. amyloliquefaciens, B. stearothermophilus, Bacillus sp. NCIB 12289, NCIB 12512, NCIB 12513 or DSM 9375, or DSMZ no.12649, KSM AP1378, or KSM K36 or KSM K38.

The Bacillus alpha-amylase may be a variant having one or more deletions in positions D183 and G184, respectively, and may further have a substitution in position N195F (using SEQ ID NO: 4 numbering). The Bacillus alpha-amylase variant may also be one having one or more deletions in position D183 and G184, and may further have one or more of the following substitutions: R118K, N195F, R320K, R458K (using SEQ ID NO: 6 numbering).

Specifically the Bacillus variant may have a double deletion in positions D183 and G184 and further comprise the following substitutions: R118K+N195F+R320K+R458K (using SEQ ID NO: 6 numbering).

The alkaline scouring enzyme(s) is(are) selected from the group consisting of: alkaline pectinase, cellulase, lipase, protease, cutinase, xyloglucanase, and mixtures thereof.

In a preferred embodiment the alkaline pectinase is a pectate lyase, preferably a pectate lyase derived from a strain of Bacillus, preferably a strain of Bacillus licheniformis, Bacillus alcalophilus, Bacillus pseudoalcalophilus, and Bacillus clarkia, especially the species Bacillus licheniformis, ATCC 14580.

Further agents suitable for the process to be performed may be added separately or be comprised in the composition of the invention. Examples of such agents include stabilizer, surfactant, wetting agent, dispersing agent, sequestering agent and emulsifying agent and mixtures thereof.

Although the alkaline alpha-amylase and alkaline scouring enzyme may be added as such, it is preferred that it is formulated into a suitable composition. Thus, the enzymes may be used in the form of a granulate, preferably a non-dusting granulate, a liquid, in particular a stabilized liquid, a slurry, or in a protected form. Dust free granulates may be produced, e.g., as disclosed in U.S. Pat. Nos. 4,106,991 and 4,661,452 (both to Novozymes A/S) and may optionally be coated by methods known in the art.

Liquid enzyme preparations may, for instance, be stabilized by adding a polyol such as, e.g., propylene glycol, a sugar or sugar alcohol or acetic acid, according to established methods. Other enzyme stabilizers are well known in the art. Protected enzymes may be prepared according to the method disclosed in EP 238 216.

In principle the composition of the invention comprising an alkaline alpha-amylase and a scouring enzyme may contain any other agent to be used in the combined process of the invention. The composition of the invention comprises in a preferred embodiment at least one further component selected from the group consisting of stabilizers, surfactants, wetting agents, dispersing agents, sequestering agents and emulsifying agents. All of such further components suitable for textile use are well know in the art.

Suitable surfactants include the ones mentioned in the “Detergent” section above. The wetting agent serves to improve the wettability of the fibre whereby a rapid and even desizing and scouring may be obtained. The emulsifying agent serves to emulsify hydrophobic impurities present on the fabric. The dispersing agent serves to prevent that extracted impurities redeposit on the fabric. The sequestering agent serve to remove ions such as Ca, Mg and Fe, which may have a negative impact on the process and preferred examples include caustic soda (sodium hydroxide) and soda ash (sodium carbonate).

Use of the Composition of the Invention

In the third aspect the invention relates to the use of the composition of the invention in a simultaneous desizing and scouring process, preferably the process of the invention. In a preferred embodiment the composition of the invention is used in a process of the invention.

Materials & Methods

Alkaline Alpha-Amylase:

Alkaline Alpha-amylase SZ is a variant alpha-amylase of the Bacillus sp. alpha-amylase backbone disclosed as SEQ ID NO: 2 in WO 00/60060. The amino acid sequence of said backbone has the following six amino acid deletions/substitutions:
   D183*+G184*+R118K+N195F+R320K+R458K.

The variant is also disclosed in WO 01/66712. The alkaline alpha-amylase was produced in batch 03AGE014-4. The enzyme is available from Novozymes A/S on request.
Alkaline Alpha-amylase NL is a variant alpha-amylase of the Bacillus sp. Alpha-amylase backbone disclosed as SEQ ID NO: 2 in WO 95/26397 derived from Bacillus sp. NCIB 12512 with a double-deletion in D183*+G184*. The alkaline alpha-amylase was produced in batch APNO0012. The enzyme is available from Novozymes A/S on request.
Pectate Iyase SP is a Bacillus licheniformis pectate lyase disclosed as SEQ ID NO: 2 in U.S. Pat. No. 6,284,524. The pectate lyase derived from Bacillus was produced in batch KND01001. The enzyme is available from Novozymes A/S on request.
   Methods:
   Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes AIS, Denmark, which folder is hereby incorporated by reference.

The Viscosity Assay APSU

APSU units: The APSU unit assay is a viscosity measurement using the substrate polygalacturonic acid with no added calcium.

The substrate 5% polygalacturonic acid sodium salt (Sigma P-1879) is solubilized in 0.1 M Glycin buffer pH 10. The 4 ml substrate is preincubated for 5 min at 40° C. The enzyme is added (in a volume of 250 microL) and mixed for 10 seconds on a mixer at maximum speed it is then incubated for 20 minutes at 40° C. For a standard curve double determination of a dilution of enzyme concentration in the range of 5 APSU/ml to above 100 APSU/ml with minimum of 4 concentrations between 10 and 60 APSU per ml.

The viscosity is measured using a MIVI 600 from the company Sofraser, 45700 Villemandeur, France. The viscosity is measured as mV after 10 sec.

For calculation of APSU units an enzyme standard dilution as described above was used for obtaining a standard curve. The GrafPad Prism program, using a non linear fit with a one phase exponential decay with a plateau, was used for calculations. The plateau plus span is the mV obtained without enzyme. The plateau is the mV of more than 100 APSU and the half reduction of viscosity in both examples was found to be 12 APSU units with a standard error of 1.5 APSU.

The Lyase Assay (at 235 nm)

For determination of the beta-elimination an assay measuring the increase in absorbance at 235 nm was carried out using the substrate 0.1% polygalacturonic acid sodium salt (Sigma P-1879) solubilized in 0.1 M Glycin buffer pH 10. For calculation of the catalytic rate an increase of 5.2 Absorbency at 235 units per min corresponds to formation of 1 micro-mol of unsaturated product (Nasuna and Starr, (1966) J. Biol. Chem., Vol. 241 page 5298-5306 (1966); and Bartling, Wegener and Olsen, Microbiology, Vol. 141 page 873-881 (1995)).

Steady state condition using a 0.5 ml cuvette with a 1 cm light path on a HP diode array spectrophotometer in a temperature controlled cuvette holder with continuous measurement of the absorbency at 235 nm. For steady state a linear increase for at least 200 sec was used for calculation of the rate. It was used for converted to formation micro-mol per min product.

Lipase Activity (LU)

The cutinase activity is determined as lipolytic activity determined using tributyrine as substrate. This method was based on the hydrolysis of tributyrin by the enzyme, and the alkali consumption is registered as a function of time.

One Lipase Unit (LU) is defined as the amount of enzyme which, under standard conditions (i.e., at 30° C.; pH 7.0; with Gum Arabic as emulsifier and tributyrine as substrate) liberates 1 micro-mol titrable butyric acid per minute. A folder AF 95/5 describing this analytical method in more detail is available upon request to Novozymes A/S, Denmark, which folder is hereby included by reference.

Determination of Cellulase Activity (ECU)

The cellulolytic activity may be determined in endo-cellulase units (ECU) by measuring the ability of the enzyme to reduce the viscosity of a solution of carboxymethyl cellulose (CMC).

The ECU assay quantifies the amount of catalytic activity present in the sample by measuring the ability of the sample to reduce the viscosity of a solution of carboxy-methylcellulose (CMC). The assay is carried out in a vibration viscosimeter (e.g. MIVI 3000 from Sofraser, France) at 40° C.; pH 7.5; 0.1 M phosphate buffer; time 30 min; using a relative enzyme standard for reducing the viscosity of the CMC substrate (Hercules 7 LFD), enzyme concentration approx. 0.15 ECU/ml. The arch standard is defined to 8200 ECU/g.

One ECU is amount of enzyme that reduces the viscosity to one half under these conditions.

The following non-limiting examples illustrate the invention.

### EXAMPLES

### Example 1

Desizing of Cotton Fabric without Enzyme

A 100% cotton woven fabric (270 g/m², fabric construction is Cupper 3/1. Warp: 28 thread/cm, and Weft: 14 thread/cm) was obtained from Boras Wafveri Kungsfors, Sweden. This fabric has 8% starch based size on its warp yarns. A fabric swatch of 0.25 m×0.5 m was cut and used. A 25 mM buffer pH 9 was made from sodium tetraborate. A 0.5 g/l surfactant BRIJ78 from Unichema and 0.5g/l surfactant Volel® TDA-7 Ethoxylate from SASOL were added in the buffer. The fabric swatch was immersed in 1 liter the surfactant-containing buffer solution for about 30 seconds and then padded through a padder (Mathis) at about 50° C. to a 90% wet pickup. The swatch was sealed immediately in a plastic bag, which was incubated at 50° C. for 1 hour. After incubation, the fabric swatch was rinsed in 90° C. water through four rinsing boxes in Mathis pad-steam range. The total time to pass the rinsing boxes was about 8 minutes. The fabric swatch was first dried in air and then equilibrated in a conditioning room at 65% relative humidity and 21° C. (70° F.) for at least 24 hours prior to analyses.

Desizing (Tegewa Method)

The starch size residue was determined visually by comparing an iodine stained fabric swatch to a standard set of photos with 1-9 scale where 1 is dark blue and 9 has no color stain. The iodine stain solution was made by dissolving 10 g KI in 10 ml water, add 0.635 g I₂, and 200 ml ethanol in deionized water to make total 1 liter solution. A fabric sample was cut and immersed in the iodine solution for 60 seconds and rinsed in deionized water for about 5 seconds. The fabric sample was rated by at least two professionals after excess water in the sample was pressed out. An average number was given. Method and standard scales obtainable from Verband TEGEWA, Karlstrasse 21, Frankfurt a.M., Germany.

Pectin Removal

The pectin residue on fabric was determined quantitatively. The principle is that ruthenium red binds to polyanionic compounds like unmethylated pectin. The level of pectin on the fabric is proportional to the concentration of ruthenium red on the cotton fabric which is linearly proportional to Kulbelka-Munk function (i.e. K/S). The color reflectance (R) of ruthenium red stained fabric was measured at 540 nm (Macbeth calorimeter, Model # CE-7000) and automatically calculated into a K/S value by:
K/S=(1-R)²/2R).

The % pectin removal was calculated by:
%-pectin removal=1-% Res. Pectin=1-100* (K/S-K/S₀)/(K/S₁₀₀-K/S₀)
where K/S₁₀₀ was from fabric with 100% pectin, typically original untreated fabric, while K/S₀ was from the fabric with 0% residual pectin, typically heavily scoured and bleached fabric. Based on information from John H. Luft and described in an article “Ruthenium red and Violet I. Chemistry” 1971, the stain solution was prepared by dissolving 0.2 g/l ruthenium red, 1.0 g/l ammonium chloride, 2.5 ml/l 28% ammonium hydroxide solution, 1.0 g/l Silwet L-77, and 1.0 g/l Tergitol 15-S-12 in distilled water to make total 1 lter solution. The solution was made daily before use. During staining, 100 mL dye solution was used for 1 gram of fabric. The fabric swatches were incubated in ruthenium red solution for 15 minutes at room temperature. The swatch was rinsed in a strainer and then rinsed in distilled water (100 ml/1 gram fabric) at 60° C. for 10 minutes. The color reflectance was measured after dry.
Fabric Wettability

Fabric wettability was measured using a drop test method according to AATCC test method 79-1995. A drop of water was allowed to fall from a fixed height (1 cm) onto the taut surface of a test specimen. The time required for the specular reflection of the water drop to disappear was measured and recorded as wetting time.

Fabric Whiteness

The desized fabric swatch was passed through a bleach bath and padded to 90-100% wet pick up. The bleaching bath contains 10 ml/L sodium silicate 40-42 Be, 5 g/l 40% EDTA, 16 ml/L 50% w/v sodium hydroxide, and 16 ml/L 50% hydrogen peroxide. The fabric swatch was incubated at 100° C. for 40 minutes, it was then rinsed in four rinsing boxes at 95° C., 65° C., 65° C. and 75° C., respectively. The total time to pass the rinsing boxes was about 8 minutes. After air dry and conditioning, fabric whiteness was measured according to AATCC test method 110-1995. The CIE tristimulus values were measured using a reflectance colormeter (Macbeth colorimeter, Model # CE-7000) with CIE illuminant D65 from 330-700 nm and 1964 10° observer. The whiteness was calculated from formulas based on the CIE chromaticity coordinates.

The test results are shown in Table 1.

### Example 2

Desizing of Cotton Fabric with an Alpha-Amylase Enzyme

The fabric and desizing process were essentially the same as in Example 1 except that Alpha-Amylase SZ of 10 KNU/L was added into the surfactant containing buffer solution prior to desizing.

The starch size residue, pectin residue, fabric wettability and whiteness were evaluated the same way as in Example 1. The test results are shown in Table 1.

### Example 3

Simultaneous Desizing and Bioscouring of Cotton Fabric

The fabric and desizing process were essentially the same as in Example 2 except that Pectate Lyase SP of 250 APSU/L was also added into the desizing solution prior to desizing.

The starch size residue, pectin residue, fabric wettability and whiteness were evaluated the same way as in Example 1. The test results are shown in Table 1.

### Example 4

Simultaneous Desizing and Bioscourina of Cotton Fabric

The fabric and desizing process were essentially the same as in Example 2 except that a Pectate Lyase SP of 750 APSU/L was also added into the desizing solution prior to desizing.

The starch size residue, pectin residue, fabric wettability and whiteness were evaluated the same way as in Example 1. The test results are shown in Table 1.

### Example 5

Simultaneous Desizing and Bioscouring of Cotton Fabric

The fabric and desizing process were essentially the same as in Example 2 except that a Pectate Lyase SP of 1500 APSU/L was also added into the desizing solution prior to desizing.

The starch size residue, pectin residue, fabric wettability and whiteness were evaluated the same way as in Example 1. The test results are shown in Table 1.

### Example 6

Desizing of Cotton Fabric with an Alpha-amylase Enzyme (NL)

The fabric and desizing process were essentially the same as in Example 1 except that Alpha-Amylase NL of 10 KNU/L was added into the surfactant containing buffer solution prior to desizing.

The starch size residue, pectin residue, fabric wettability and whiteness were evaluated the same way as in Example 1. The test results are shown in Table 1.

### Example 7

Simultaneous Desizing and Bioscouring of Cotton Fabric

The fabric and desizing process were essentially the same as in Example 6 except that Pectate Lyase SP of 250 APSU/L was also added into the desizing solution prior to desizing.

The starch size residue, pectin residue, fabric wettability and whiteness were evaluated the same way as in Example 1. The test results are shown in Table 1.

### Example 8

Simultaneous Desizing and Bioscouring of Cotton Fabric

The fabric and desizing process were essentially the same as in Example 6 except that Pectate Lyase SP of 750 APSU/L was also added into the desizing solution prior to desizing.

The starch size residue, pectin residue, fabric wettability and whiteness were evaluated the same way as in Example 1. The test results are shown in Table 1.

### Example 9

Simultaneous Desizing and Bioscourina of Cotton Fabric

The fabric and desizing process were essentially the same as in Example 6 except that Pectate Lyase SP of 1500 APSU/L was also added into the desizing solution prior to desizing.

The starch size residue, pectin residue, fabric wettability and whiteness were evaluated the same way as in Example 1. The test results are shown in Table 1.

### Example 10

Desizing of Cotton Fabric with Alpha-Amylase SZ

The fabric and desizing process were essentially the same as in example 1 except that Alpha-Amylase SZ of 50 KNU/L was added into the surfactant containing buffer solution prior to desizing.

The starch size residue, pectin residue, fabric wettability and whiteness were evaluated the same way as in Example 1. The test results are shown in Table 1.

### Example 11

Simultaneous Desizing and Bioscouring of Cotton Fabric

The fabric and desizing process were essentially the same as in Example 10 except that Pectate Lyase SP of 250 APSU/L was also added into the desizing solution prior to desizing.

The starch size residue, pectin residue, fabric wettability and whiteness were evaluated the same way as in Example 1. The test results are shown in Table 1.

### Example 12

Simultaneous Desizing and Bioscouring of Cotton Fabric

The fabric and desizing process were essentially the same as in Example 10 except that Pectate Lyase of 750 APSU/L was also added into the desizing solution prior to desizing.

The starch size residue, pectin residue, fabric wettability and whiteness were evaluated the same way as in Example 1. The test results are shown in Table 1.

### Example 13

Simultaneous Desizing and Bioscouring of Cotton Fabric

The fabric and desizing process were essentially the same as in Example 10 except that Pectate Lyase SP of 1500 APSU/L was also added into the desizing solution prior to desizing.

The starch size residue, pectin residue, fabric wettability and whiteness were evaluated the same way as in Example 1. The test results are shown in Table 1.

TABLE 1

| | | Pectin | | Whiteness |
|---|---|---|---|---|
| | Desizing | Removal | Wetting time | after bleaching |
| Example # | (Tegewa) | (%) | (seconds) | CIE Ganz82 |
| 1 | 3.0 | 11.0 | 6.4 | 63.0 |
| 2 | 4.75 | 13.9 | 13.6 | 64.2 |
| 3 | 4.5 | 28.5 | 7.3 | 64.3 |
| 4 | 4.5 | 45.8 | 3.4 | 65.4 |
| 5 | 4.75 | 52.3 | 2.3 | 64.7 |
| 6 | 4.25 | 14.7 | 8.8 | 65.5 |
| 7 | 4.5 | 25.7 | 6.4 | 65.3 |
| 8 | 4.5 | 42.1 | 4.9 | 65.1 |
| 9 | 4.0 | 49.5 | 4.4 | 65.4 |
| 10 | 5.5 | 17.4 | 6.6 | 66.2 |
| 11 | 6.25 | 31.9 | 4.0 | 66.6 |
| 12 | 6.25 | 45.2 | 3.1 | 67.3 |
| 13 | 5.75 | 50.9 | 3.0 | 67.6 |

1-34. (canceled)

35. A process for simultaneously desizing and scouring of sized fabric containing starch or starch derivatives, which process comprises treating the fabric with an alkaline alpha-amylase and an alkaline pectate lyase.

36. The process of claim 35, wherein the alkaline pectate lyase is derived from a strain of Bacillus.

37. The process of claim 35, wherein the pectate lyase is the mature pectase lyase in SEQ ID NO: 2.

38. The process of claim 35, wherein the alkaline alpha-amylase is derived from a Bacillus sp.

39. The process of claim 35, wherein the alkaline alpha-amylase has an amino acid sequence of SEQ ID NO: 4 or SEQ ID NO: 6.

40. The process of claim 35, wherein the alpha-amylase is a Bacillus alpha-amylase variant which comprises one or more deletions in positions D183 and G184 (using SEQ ID NO: 4 numbering).

41. The process of claim 40, wherein said Bacillus alpha-amylase variant further comprises the substitution N 195F.

42. The process of claim 40, wherein the Bacillus alpha-amylase variant further comprises one or more of the following substitutions: R118K, N195F, R320K, and R458K.

43. The process of claim 35, wherein the alkaline alpha-amylase is present in a concentration of 0.05-150 KNU/L treating solution or 0.05-150 KNU/Kg fabric.

44. The process of claim 35, wherein the pectate lyase is present in a concentration in the range from 1-1,500 APSU/kg fabric.

45. The process of claim 35, wherein the process is carried out at a pH in the range of 7-11.

46. The process of claim 35, wherein the process is carried out at a temperature in the range from 30 to 115° C.

47. The process of claim 35, wherein the process is carried out in the presence of a surfactant.

48. The process of claim 35, wherein the fabric is a cellulosic fabric.

49. The process of claim 35, wherein the fabric is a silk fabric or a wool fabric.

50. The process of claim 35, wherein the fabric is a polyester containing fabric or garment consists of essentially 100% polyester.

51. The process of claim 50, wherein the polyester fabric is a polyester blend, such as a polyester and cellulosic blend, including polyester and cotton blends; a polyester and wool blend; a polyester and silk blend; a polyester and acrylic blend; a polyester and nylon blend; a polyester, nylon and polyurethane blend; a polyester and polyurethane blend, rayon (viscose), cellulose acetate and Tencel.

52. The process of claim 35, wherein the process is carried out in a single bath.

53. A composition comprising an alkaline alpha-amylase and an alkaline scouring enzyme.

54. The composition of claim 53, wherein the alkaline alpha-amylase is derived from a Bacillus sp.

55. The composition of claim 53, wherein the scouring enzyme is selected from the group consisting of alkaline pectinase, cellulase, lipase, protease, cutinase, xyloglucanase, and mixtures thereof.

56. The composition of claim 54, wherein the scouring enzyme is a pectate lyase.

57. The composition of claim 53, wherein the alpha-amylase is a Bacillus alpha-amylase comprising one or more deletions in position D183 and G184.

58. The composition of claim 57, wherein the Bacillus alpha-amylase further comprise one or more of the following substitutions: R118K, N195F, R320K, and R458K.

59. The composition of claim 53, wherein the composition further comprises one or more of stabilizers, surfactants, wetting agents, dispersing agents, sequestering agents and emulsifying agents.
